(19) United States
(12) Patent Application Publication
(10) Pub. No.: US 2018/0197633 A1
(43) Pub. Date: Jul. 12, 2018

(54) SYSTEMS AND METHODS FOR TREATMENT OF STROKE

(71) Applicant: Penumbra, Inc., Alameda, CA (US)

(72) Inventor: Brijesh P. Mehta, Miami Lakes, FL (US)

(73) Assignee: Penumbra, Inc., Alameda, CA (US)

(21) Appl. No.: 15/917,308

(22) Filed: Mar. 9, 2018

Related U.S. Application Data
(63) Continuation-in-part of application No. 15/356,248, filed on Nov. 18, 2016.
(60) Provisional application No. 62/257,400, filed on Nov. 19, 2015.

(51) Int. Cl. G16H 40/20; A61B 5/00; A61B 5/11; G06Q 10/06; G16H 70/20

(52) U.S. Cl. CPC G16H 40/20; A61B 5/0022; A61B 5/1113; A61B 5/74; G16H 70/20; A61B 5/681; G06Q 10/0633

(57) ABSTRACT

A system for treatment of ischemic stroke provides a stroke treatment workflow plan defining series of diagnostic actions and therapeutic actions to be performed at locations within a health care facility identified by beacons detectable by proximity sensors that travel with the patient. A first communications device having wireless communications capabilities receives a signal from a proximity sensor and typically transmits data to a second communications device having a visible timer and configured to receive data from and send data to other wireless communications devices. When the a patient undergoes diagnosis and treatment via the workflow plan, the system tracks the location of the patient within the workflow plan and the time at which the patient is at each location, and records the location of the patient and the time of the location within the workflow plan.

### CROSS-REFERENCE TO RELATED APPLICATIONS

This application is a continuation-in-part of U.S. patent application Ser. No. 15/356,248, (Attorney Docket No. 41507-718.201), filed Nov. 18, 2016, which claims the benefit of U.S. Provisional Application No. 62/257,400 (Attorney Docket No. 41507-718.101), filed Nov. 19, 2015, the entire content of which are incorporated herein by reference.

### FIELD OF THE INVENTION

The present invention relates generally to medical systems and procedures, and management of medical procedures and personnel. Specifically, the invention relates to a system for diagnosis and treatment of disease. The invention further relates to methods of administering technology intensive medical care and managing multidisciplinary teams that perform complex, life-saving medical procedures within restrictive time constraints. The invention also relates to business methods for evaluating a treatment facility's effectiveness in handling complex medical procedures, and for providing quantified consultation to a treatment facility for improvement of the care provided by the facility.

### BACKGROUND OF THE INVENTION

Stroke is a significant cause of disability and death, and is a growing problem for global healthcare. More than 700,000 people in the United States alone suffer a stroke each year, and of these, more than 150,000 people die. Of those who survive a stroke, roughly 90% will suffer long term impairment of movement, sensation, memory, or reasoning, ranging from mild to severe. The total cost to the U.S. healthcare system is estimated to be over $50 billion per year, and, adding indirect costs, stroke is estimated to cost the U.S. more than $70 billion per year.

Stroke may be caused from a rupture of a cerebral artery (referred to as a “hemorrhagic stroke”), or by a blockage or occlusion in a cerebral artery resulting from a thromboembolism (referred to as an “ischemic stroke”). Roughly 80% of strokes are classified as ischemic. When a patient experiences an ischemic stroke, the occlusion prevents blood flow to vital brain tissue, thereby depriving the tissue of oxygen, causing nerve cell damage and potentially cell death. Among patients experiencing a stroke due to a large vessel occlusion, approximately 1.9 million nerve cells (or neurons) are at risk for irreversible injury every minute that elapses, until blood flow is restored. Providing rapid and effective diagnosis and treatment of stroke is therefore vital for protecting and restoring patient health.

Health education aims to alert the public to the signs and symptoms of stroke, and to the vital importance of getting immediate medical assistance when a stroke is suspected. Once medical assistance is sought, either through an emergency response by paramedics or upon arrival in a hospital emergency room, a series of examinations and tests is initiated. Stroke diagnosis begins with a patient interview and examination, utilizing established protocol to detect one-sided weakness or paralysis, speech difficulty, or other common symptoms of stroke. In order to objectively quantify the impairment caused by a stroke, a healthcare provider will use the National Institutes of Health Stroke Scale, or NIHSS. A protocol including 11 items of inquiry, the sum of the patient's score for each inquiry is calculated in order to assign a score reflecting the severity of the stroke. Further, if a stroke is suspected as a result of the interview and physical exam, then diagnostic imaging such as CT scan, MRI, ultrasound, or some combination is performed in order to definitively diagnose a stroke. The imaging process also determines the location of an occlusion, and prepares clinicians for treating the clot. All of the foregoing requires the recording of information and the communication of test results to treating physicians. Therefore, beginning with emergency responders, admissions personnel, physicians, nurses, diagnostic imaging technicians, and other support personnel, the diagnostic process alone involves numerous medical professionals, and requires rapid and precise communication of results throughout the protocol, all accompanied by the time pressure presented in a case of acute stroke.

In a case of diagnosis of acute ischemic stroke, there are currently two FDA approved therapies: intravenous administration of a drug referred to as tissue plasminogen activator, or tPA, and mechanical thrombectomy performed under fluoroscopic imaging. Approved use of tPA is limited to within three hours of symptom onset, while mechanical thrombectomy may be deployed within up to eight hours or more. In view of the time constraints for safe administration of tPA and the rapid loss of neurons suffered during stroke, the American Stroke Association (ASA) guidelines recommend administration of intravenous tPA within 60 minutes of time of arrival at the hospital.

If intravenous tPA is not an option for treatment, mechanical thrombectomy may be the desired course of therapy. Mechanical thrombectomy typically involves the use of an intravascular device such as a catheter. The distal end of an interventional catheter is introduced via a remote incision site (typically in the groin), and tracked to the site of the occlusion. The therapy typically involves aspiration, and may utilize additional interventional devices, such as a clot remover or separator, which is mounted to the distal end of the catheter. Recent randomized control trials show that rapid reperfusion is associated with favorable clinical outcomes. (See Stroke 2015; 46:3020-3035). Formulated from these studies are goal times of “picture-to-puncture” in less than 60 minutes, and “door-to-device” within 90 minutes. Needless to say, diagnosing and administering stroke treatment within these guidelines requires efficient work and communication among a large team of medical professionals, including emergency physicians, nurses, neurologists, radiologists, neurointerventionalists, and catheter lab staff. In order to consistently perform at a high level in delivery of acute stroke therapies and remain compliant with the ASA/Joint Commission recommendations, renewed focus has been placed on tracking time intervals during the in-hospital stroke processes in order to evaluate and to optimize workflows.

However, stroke treatment coordinators and quality improvement specialists currently manually track specified treatment metrics, and only through retrospective data extrapolation from Electronic Health Records (EHR) and from paper charts. Existing hardware and software solutions available in the market still require significant data entry during the time-sensitive stroke treatment procedures. These methods of data collection are disruptive, time consuming, susceptible to error, and lack the instructive benefit of rapid feedback following the conclusion of a case. Consequently, these methods are less than advantageous to continuous process improvement. Other attempts to improve the speed with which hospitals diagnose and treat stroke include pre-hospital notification from emergency personnel to stroke centers, but do not provide a comprehensive solution to the challenges presented. There remains a need to effectively track, log, and analyze protocol metrics. Moreover, there remains a need to establish and enhance immediate communication of test and imaging results among all team members simultaneously.

The aforementioned shortcomings in the prior art are also applicable to treatment of conditions other than stroke. For example, many of the same needs arise in the context of treatment of cardiac arrest, myocardial infarction, epilepsy, and childbirth. Therefore, a desirable solution to stroke treatment may have significant utility in many other treatment contexts.

A desirable solution should be easy to implement, and customizable to fit the hospital's existing procedures. The system should be HIPAA compliant, secure, and include reliable, automated capture of checkpoint metrics. The system and methods should provide automatic reporting of key data obtained during diagnosis and treatment.

### BRIEF DESCRIPTION OF THE DRAWINGS

FIG. 1 is a flow chart illustrating a series of exemplary tasks in stroke diagnosis and treatment.

FIG. 2 is a schematic timeline reflecting American Stroke Association (ASA) timing guidelines for diagnosis and treatment of stroke.

FIG. 3 is a schematic illustration of examples of devices employed in systems and methods according to the invention.

FIG. 4 is a schematic illustration of examples of devices employed in systems and methods according to the invention.

FIG. 5 is a schematic illustration of examples of devices employed in systems and methods according to the invention.

FIG. 6 is a schematic illustration of a system and method according to the invention, displayed along a proposed stroke workflow timeline.

### DETAILED DESCRIPTION OF THE INVENTION

Some embodiments of the invention are described below. For clarity, not all features of each actual implementation are described in this specification. In the development of an actual system, some modifications may be made that result in an embodiment that still falls within the scope of the invention.

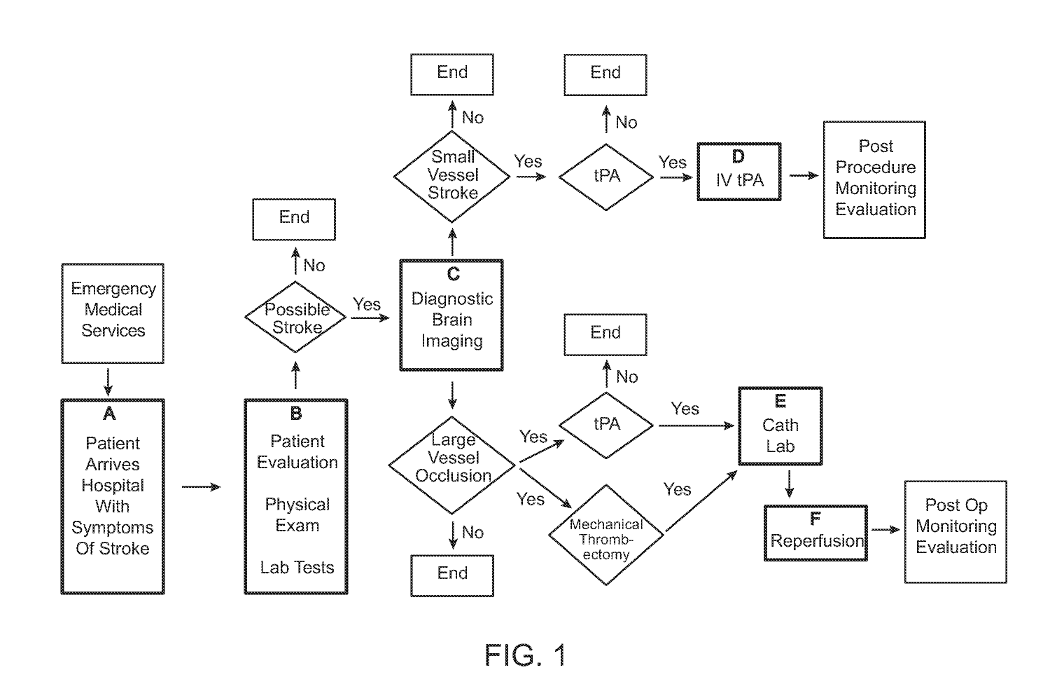

FIG. 1 is a flow chart that highlights some of the key actions that are taken during stroke diagnosis and treatment. Conceptually the decisions and actions form a workflow. The term “stroke treatment workflow” or “stroke workflow” are used herein to refer to a progressive series of decisions made and actions taken in order to diagnose and treat stroke. As can be seen in FIG. 1, the stroke workflow plan proceeds in an algorithmic fashion. That is, the workflow includes step wise instructions and decisions performed in a prescribed sequence, in order to achieve the goals of diagnosis and treatment. It will be understood that FIG. 1 does not include the finer details of diagnosis and treatment. In fact, the events represented in FIG. 1 are very general, and could be broken down into multiple actions or checkpoints that take place within the workflow. For example, the physical exam includes numerous tasks, often undertaken by more than one care provider. The physical exam generates numerous data points which in turn are placed into a diagnostic matrix or algorithm. Similarly, the patient interview may include small tasks performed by a patient (such as, for example, raising both arms), taking a medical history, and other detailed tasks. The actions reflected in FIG. 1 are greatly simplified for the purposes of demonstration and clarity.

Further, it will be understood that while the example of FIG. 1 is focused on stroke treatment, a comparable treatment workflow may be useful in diagnosing and treating other conditions, by substituting some of the key parameters and steps in stroke treatment with the key steps in the protocol for treating other conditions. Some of these key steps are referred to generically as “intervention”, “interventional measures”, “therapeutic intervention”, or comparable term. And finally, it will be understood that FIG. 1 reflects a visual representation of a stroke workflow, the terms “workflow” and “workflow plan” may refer to an electronic, computerized, algorithm, software or hardware configuration that may both represent the decision making process and be used as a tool in the decision making process of diagnosing and treating stroke. In other words, the process represented visually in FIG. 1 may be captured electronically, and the electronic version may also be referred to as a workflow.

Beginning at the far left of the figure, the first event in the workflow is referred to as “Emergency Medical Services”. During this first event, emergency responders such as, for example, paramedics respond to an emergency call for medical assistance. Emergency medical service providers evaluate the patient and, if warranted, transport the patient to a hospital. During the evaluation by emergency medical services, several data points are generated related to the patient and the patient's symptoms. (It will be noted however that in some instances, a patient is transported directly to a hospital without the intervention of emergency medical services.) Following this optional initial intervention, box A in the workflow illustrated represents the patient's arrival at a hospital. When a patient arrives at a hospital and is suffering symptoms of stroke, a series of events is initiated in order to diagnose and treat stroke. This series of events is labeled B in FIG. 1. As illustrated in B, the patient is interviewed, and a physical exam is administered that includes tests for neurological deficit. In addition, blood is drawn and laboratory tests are performed in order to detect indicators of stroke. If acute ischemic stroke is suspected, the process continues to C in the illustration of FIG. 1, and the patient undergoes diagnostic imaging tests such as a CT scan or MRI. The imaging conclusively determines whether there is an occlusion of blood flow, and if so, locates the occlusion, and reveals additional diagnostic details of the occlusion which are important for formulating a treatment plan. Importantly, the imaging determines whether the occlusion is a “Small Vessel Stroke”, or a “Large Vessel Occlusion”, both of which are noted as options in FIG. 1. If it is determined that neither of these conditions exists, for the purposes of the illustration, the workflow ends.

If a “Small Vessel Stroke” is diagnosed, a determination is made whether the patient is a candidate for intravenous tPA. If tPA is selected as the optimal treatment, the patient is prepared and if necessary, moved to a suitable location for the administration of tPA, represented by box D in FIG. 1. If, however, it is determined that administration of tPA is not within safe time limits, or is otherwise contraindicated, then tPA will not be administered, and for the purposes of the illustration of FIG. 1, the workflow ends.

If a “Large Vessel Occlusion” is diagnosed, a determination is also made whether the patient is a candidate for intravenous tPA. However, in the case of a “Large Vessel Occlusion”, a determination is also made whether the patient will undergo mechanical thrombectomy. If mechanical thrombectomy is a desired course of treatment, the patient proceeds to a catheterization lab, or “cath lab”, and is prepared for a percutaneous catheter procedure under fluoroscopic visualization (E). Mechanical thrombectomy and intravenous tPA may be administered in combination. Further, mechanical thrombectomy may involve one or more various alternative devices and methods. The goal of all of the available devices and methods is reperfusion of the affected vessel, and restoration of blood flow (F). Following reperfusion, the patient is continually monitored and evaluated. Recovery benchmarks are recorded and analyzed.

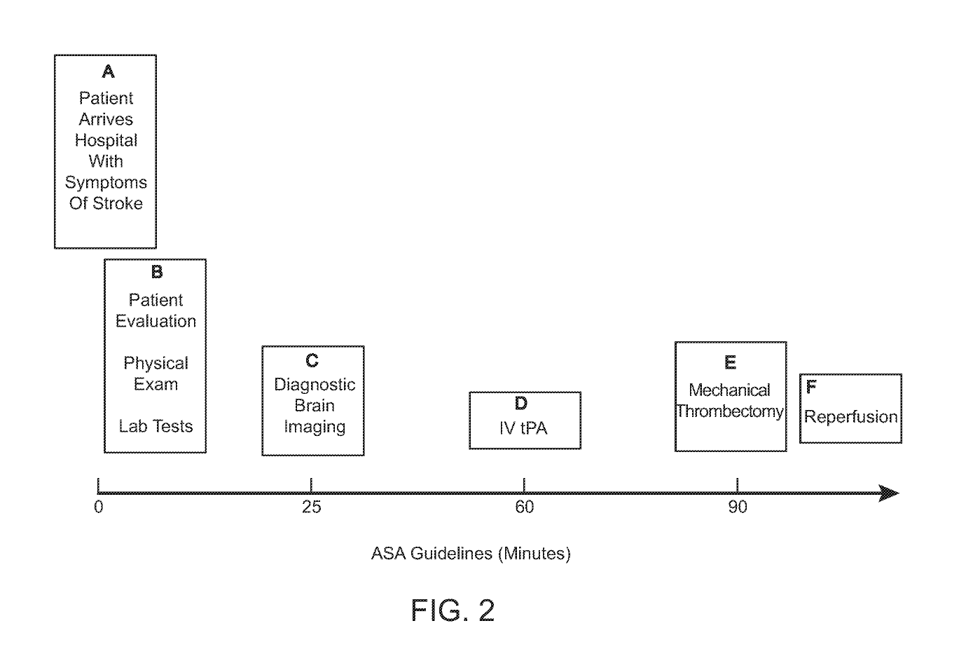

FIG. 2 is a schematic illustration of a timeline of some of the key events of FIG. 1. The points along the axis of FIG. 2 reflect the desired goal times by which it is desirable, according to American Stroke Association (ASA) guidelines, to achieve some of the major workflow tasks illustrated in FIG. 1. Beginning at the left hand side of FIG. 2, the time of arrival at the hospital (A) is considered the start time, or 0 minutes. This start point is also nicknamed “Door” time in stroke treatment protocol jargon. In the following minutes, the numerous diagnostic tasks (prior to imaging) are performed (B). Diagnostic brain imaging (C) is ideally performed within 25 minutes of arrival. And following conclusive imaging of an acute stroke, administration of intravenous tPA (D), is ideally initiated within 60 minutes of hospital arrival. Also according to the stroke guidelines discussed above, 90 minutes is the goal by which mechanical thrombectomy is performed (E), followed by reperfusion (F). Patient monitoring, periodic neurochecks and the computation of NIHSS scores continue in the hours following treatment. Diagnosis and treatment of diseases other than stroke may utilize a comparable timeline, but may include other key timing guidelines associated with key procedural steps that are appropriate for the particular disease, condition, or medical event.

The invention herein includes systems and methods for treatment of stroke, myocardial infarction, cardiac arrest, or other emergency medical treatment. The system includes a treatment workflow plan, and an interrelated group of devices and methods designed to be integrated into the workflow, with the goal of accomplishing critical tasks within the timing guidelines illustrated in FIG. 2 or other applicable timing guidelines. The invention disclosed herein provides automated tracking of a patient's progress through the workflow of FIG. 1, and provides real-time updates to all of the multidisciplinary team members as the patient progresses through the stroke treatment workflow, while continually tracking actual elapsed time. In addition, the system furnishes “push” notifications to various team members as the patient progresses through the protocol, summoning members to corresponding work stations, and alerting members to particular action items. Further, the system manages the substantial data that is generated at each point in the protocol, up to and including the conclusion of the case, thereby providing immediate feedback to the multidisciplinary team. The system incorporates machine learning, such that the branch points and nodes of the algorithm for emergency medical services triage and catheter lab activation are refined in real time, based on its backend analytics platform. Still further, the system compares the data with previous cases, immediately highlighting bottlenecks in the workflow, thereby focusing and streamlining efforts of the hospital to improve workflows. The system may even compare cases handled by competitor hospitals, and provide quantitative success rates that hospitals may use to promote their services. And still further, the system automatically exports data to spreadsheets and electronic health records (EHRs), enabling evaluation of the data and improvement of workflow efficiencies. Finally, the system includes a dashboard available online and through a mobile app providing immediate process summary upon completion of each stroke case, highlighting achievements and areas for improvement.

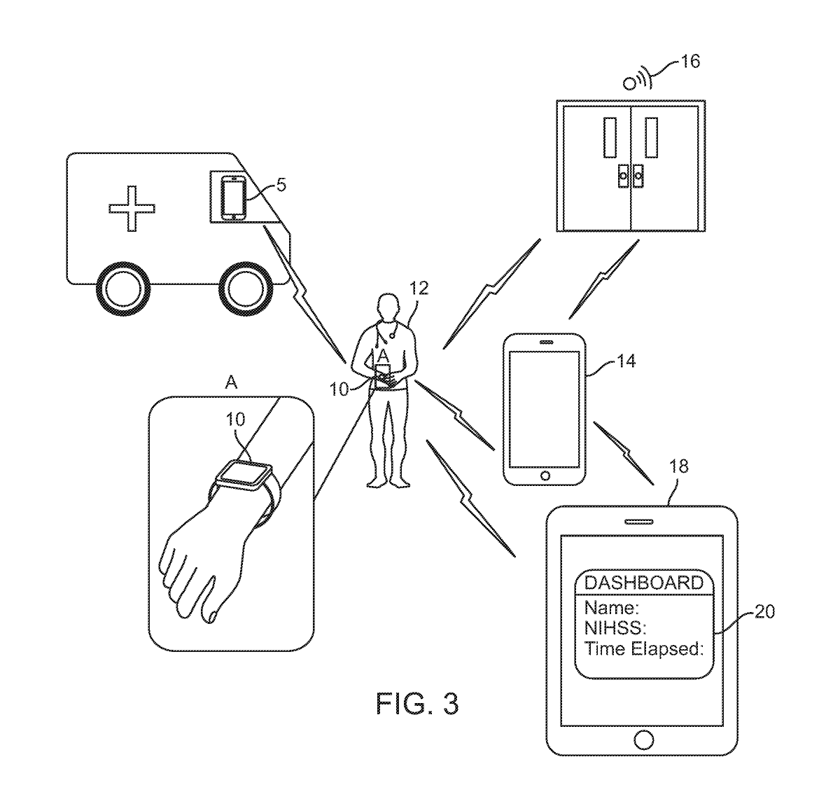

The systems and methods according to the invention incorporate known devices, and employ hardware and software customized for the system. Examples of principle devices suitable for use with the invention are illustrated in FIG. 3, and begin with a first “smart” device 5, located within an emergency medical services vehicle, representing at least one communications device used by first responders. The term “smart” device is intended to refer to a mobile communications device that utilizes an advanced mobile operating system which combines features of a personal computer operating system with communications capabilities, WiFi connectivity, the ability to accept sophisticated applications, other features useful for mobile or handheld use, and, optionally, high resolution touch screen display. A smart device may be a smart watch, phone, computer tablet, laptop computer, speaker/network home, or other communications device. Numerous brands of smart devices, such as Apple, Android, Samsung, and others are currently commercially available, and additional smart devices will become commercially available, and are suitable for use with the invention. Smart device 5 may be configured with or otherwise compatible with emergency services software, such as, for example, ESO. Diagnostic information obtained by emergency services personnel and general information, such as patient identification, estimated hospital arrival time, and other data, may be transmitted to other communications devices used in the system, as described below. Additionally, images may be scanned and seamlessly delivered to the devices for immediate evaluation. It will be understood that most all of the smart devices having comparable capabilities and utilized according to the invention are interchangeable with one another, and that the specific smart devices listed are merely examples. The particular smart device employed at particular points in a workflow will be selected based upon individual preferences and system compatibilities.

An additional key device in the illustration of FIG. 3 is a “smart” watch 10. The term “smart watch” is used herein to refer to a computerized mobile device that provides timekeeping and extensive additional functions, has the capability to run mobile applications, may have touch screen capabilities, and is designed to be worn on the wrist. Several brands of smart watches are currently commercially available, including Apple and Samsung, and numerous will become available, that are suitable for use with the invention. In the example of FIG. 3, smart watch 10 is worn by a clinician who has been charged with coordinating a multidisciplinary team of personnel that is working together on the diagnosis and treatment of a patient. The clinician may be referred to, for example, as a stroke charge nurse 12. Stroke charge nurse 12 wears smart watch 10, which is configured to receive an alert from emergency responders. Emergency personnel transmit an alert, via smart device 5, that a patient suspected of suffering a stroke is in route to the hospital. Smart watch 10, equipped with stroke workflow management software according to the invention, receives the alert. Stroke charge nurse 12, upon receiving the notification via smart watch 10, activates stroke workflow management software, alerts additional hospital personnel, and assembles a stroke treatment team. Some members of the team may additionally wear smart watches configured with stroke workflow plan software, and/or may utilize smart phones, such as, for example, smart phone 14, that are in electronic communication with smart watch 10.

Optionally, an embodiment of a system described herein may include one or more signal beacons, such as beacon 16, placed at one or more predetermined, strategic physical locations identified in the stroke workflow plan, where a patient is taken, and where one or more diagnostic or treatment tasks are performed. It should be noted that this device is suitable in an embodiment according to the invention, but is merely optional, and that alternative embodiments do not require a beacon 16. The term “beacon” is used herein to refer to an electronic, signal emitting proximity sensor, the beacon equipped to emit a unique identifier that is received by a mobile communications device (such as, for example, smart watch 10) having compatible software. An example of suitable beacons are iBeacons, (a protocol standardized by Apple, https://developer.apple.com/ibeacon/) which use Bluetooth Low Energy (LE) proximity sensing to transmit a universally unique identifier that is picked up by a compatible app or operating system. Alternative to being fixedly placed at a predetermined location in a facility, a “beacon” may be contained in a beacon “wand” that is passed in close proximity to a radiofrequency identification tag or other signal-emitting device. “Beacon” may also include or alternatively refer to radiofrequency identification tags, both transmitting and receiving, used for tracking the movement of items or persons.

An additional device that may be part of a system according to the invention is computer tablet 18. (Alternatively, or in addition to computer tablet 18, alternative electronic communications devices may be used for the illustrated purpose, such as, for example, a miniature tablet, a laptop computer, a desktop computer, a “smart TV”, a “smart speaker”, or comparable devices, not pictured.) In the example illustrated in FIG. 3, smart watch 10 and smart phone 14 can transmit data to computer tablet 18. Computer tablet 18 is equipped with compatible software, and is configured to display a “dashboard” 20. The term dashboard is used herein to refer to a software-based control panel for the applications used by the system. The dashboard may display data, both singularly and in graph or chart form, including time elapsed, actions needed, and other desired interactive elements. Computer tablet 18 may also receive and display scanned images, such as, for example, a CT scan. Stroke charge nurse 12, via smart watch 10, transmits the stroke alert and patient information to a computer tablet 18.

Additional devices that may be incorporated into the system include additional smart watches, which may be worn by medical personnel, or a patient or both; laptop computers; a “smart TV”, such as Apple TV (not pictured), one or more optional “transmission” buttons (described below), and, also optionally, a “smart speaker”, such as one or more voice activated network command center devices, such as, for example, the Apple HomePod, Amazon Echo, or comparable device (described below). A system or method according to the invention may employ any number of the aforementioned devices that are capable of receiving, transmitting, displaying and recording data, via passive signal transmission, key entry, push buttons, voice commands, or any combination thereof. The aforementioned devices are collectively referred to herein as “communications devices”, “wireless communications devices”, or “smart devices”. Moreover, many of the mentioned communications devices may be interchangeable with one another within the systems and methods disclosed herein.

In an alternative embodiment (not pictured), a radiofrequency identification tag may be included in a patient wrist bracelet, or otherwise closely associated with the patient. An example of a radiofrequency identification tag is of a type used in athletics for tracking the movement of an athlete, or used by commercial carriers to track movement of a shipped package. The identifier can be used to determine the physical location of a device (here, a patient), or trigger a location-based action. Beacons (or radiofrequency signal emitters) such as beacon 16, are located at or near the entry and/or exit of any of a number of designated sites within a hospital that are locations to which a patient is brought during stroke treatment. These sites may include an emergency room, a CT scan, MRI, or comparable imaging suite, a cath lab, and other locations. A smart watch or radiofrequency identification tag communicates wirelessly with beacons 16. Smart watch 10 in turn can communicate this information, or transmit this data, to smart phone 14. Smart phone 14 can in turn upload the information to another smart watch (not pictured), another smart phone (not pictured), a tablet 18, a smart TV (not pictured) and/or any device that may display scan images (not pictured), and online dashboard 20.

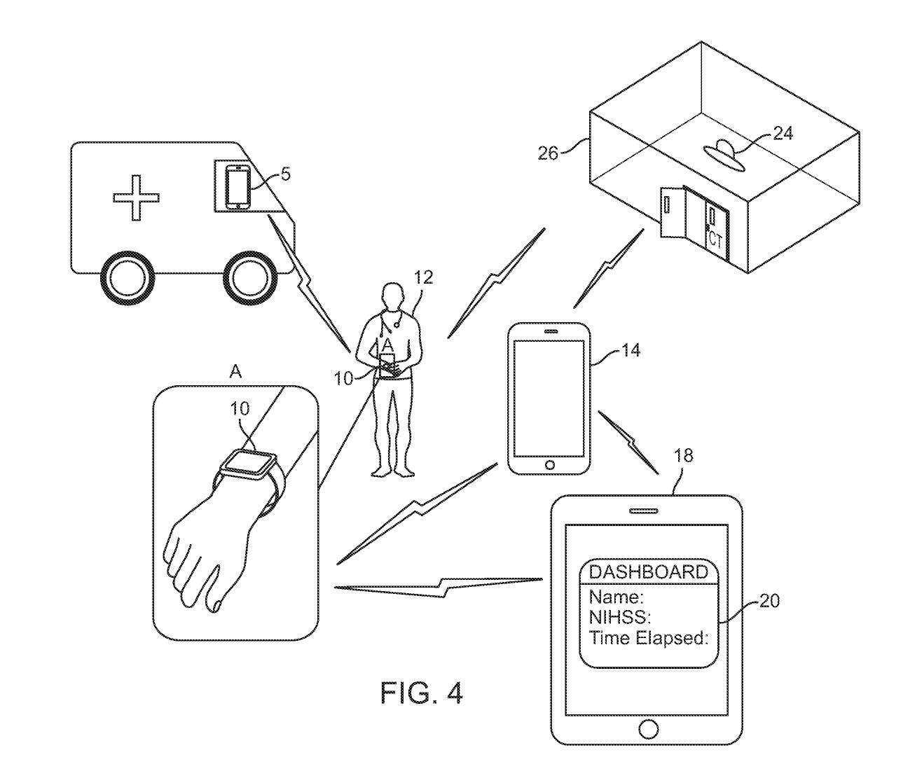

FIG. 4 illustrates an alternative embodiment according to the invention. The embodiment illustrated in FIG. 4 is very similar to that illustrated in FIG. 3, except for the inclusion of an alternative optional device. In the example of FIG. 4, instead of the signal beacon 16 of FIG. 3, an optional transmission button 24 may be incorporated into the system. Like beacon 16, transmission button 24 is suitable for use in an embodiment according to the invention, but not all embodiments require either beacon 16 or transmission button 24. The term “transmission button” is used to refer to a small electronic communications device that is configured or programmed to send a specific communication signal simply by manually pressing the button. Transmission button 24 may be equipped with software that is programmed to transmit a specific signal to a smart phone or other communications device, the signal indicating that a particular task has been performed. In the system described herein, transmission button 24 is located in or near a location where CT scans are performed. In this example, transmission button 24 is located within imaging suite 26. Additional transmission buttons may alternatively or additionally be located within or near other locations as customized by a particular treatment center and the center's associated workflow plan. In any event, when pressed manually by a clinician, transmission button 24 communicates wirelessly with smart watch 10 and smart phone 14, to transmit a unique signal that is associated with a defined task or checkpoint within the stroke workflow. Transmission button 24 communicates to, for example, register patient location with respect to contextual steps in the workflow, and to log specific time points, such as CT scan completion, initiation of tPA administration, etc. This data is transferred to smart watch 10 and/or smart phone 14, to continue the tracking of critical information regarding the patient's test results, overall condition, and the patient's progress through the stroke treatment workflow.

The system preferably includes turnkey hardware/software. The software preferably is user friendly, includes a simple user interface and requires minimal lead-in training. It must be HIPAA compliant, secure, and utilize data encryption. The smart watch 10 and smart phone 14 permit rapid data entry, (e.g., patient age/name, NIHSS score, etc.), by physicians and nurses through a simple user interface during the treatment workflow. The system should include the ability to receive and display images such as, for example, CT scans, share case summary and dashboard metrics with emergency management systems (EMS) as part of a virtual poster; to compare process metrics with other sites utilizing the platform around the world; and backend data analytics software for quality improvement and research. Optionally, a system according to the invention may comprise software configured to perform machine learning, described more fully below.

The system further includes a stroke process app designed for a smart phone 14 that displays time lapse, and also receives information (such as patient location, etc.) via the smart watch 10, as the patient progresses through the stroke treatment process pathway. The smart phone 14 (such as, for example, an iPhone 6 plus), may be stationed on the patient stretcher, permitting team members to view time lapse from arrival at the hospital, and to input data. The highly visible display of time lapse conveys the continuing sense of urgency throughout the protocol. The various phases and time intervals of the workflow can also be displayed as each step in the process is completed, keeping all team members aware of the patient's progress and the hospital's efficiency. Location specific features in the smart phone software will allow entry of contextual data such as age, NIHSS score, Last Seen Well (LSW) time, tPA administration time, puncture time/devices used/reperfusion time/thrombolysis in cerebral infarction (TICI) score, via beacons prompting next steps along the workflow programmed into the app. All fields should be easily customizable based on hospital preferences.

The smart watch 10 (such as, for example, Apple Watch) is programmed to display workflow specific checkpoints, which stroke charge nurse 12 acknowledges with the touch of a finger, to input patient progress data into the system. The smart watch 10 may additionally behave as a key that unlocks each phase of the stroke process on the smart phone 14 after receiving pings from stroke charge nurse 12, or beacon 16, transmission button 24, or some combination of the foregoing.

The system may also incorporate a mobile app for smart phones used by treating physicians and nurses, who would receive push notifications of the stroke workflow as the patient progresses through the process. And upon completion of the stroke case, the data would be pushed immediately to an online dashboard, with options to export to the hospital's EHR for seamless documentation.

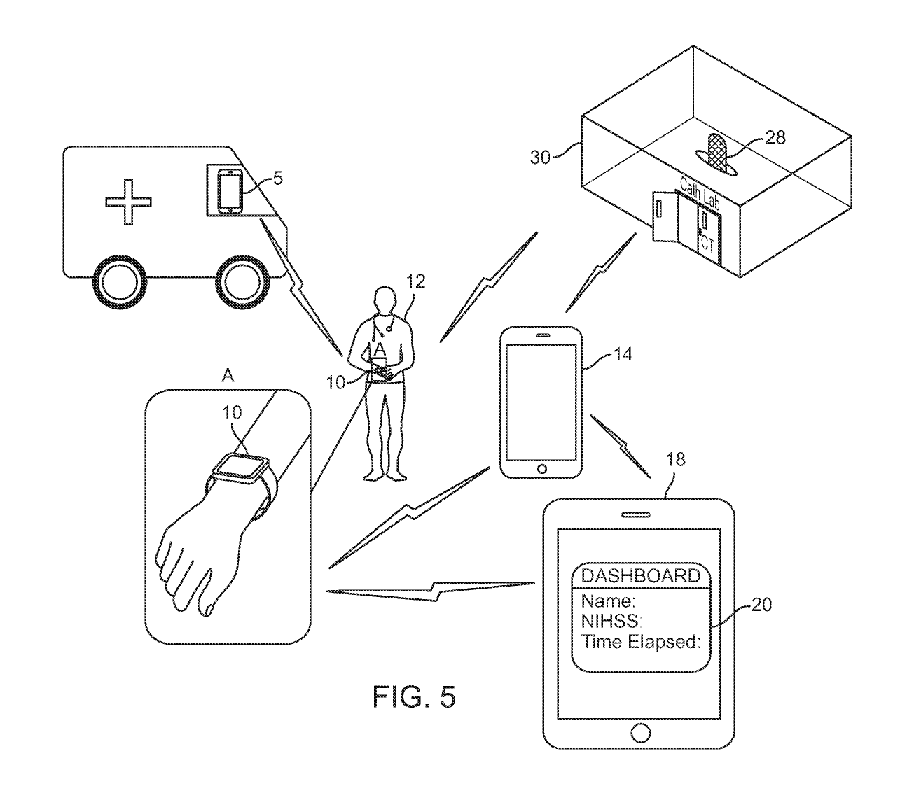

Turning now to FIG. 5, yet another alternative embodiment according to the invention is illustrated. In the example of FIG. 5, optional signal beacon 16 of FIG. 3 or optional transmission button 24 of FIG. 4 have been replaced by optional voice activated smart device 28. Like signal beacon 16 and transmission button 24, voice activated smart device 28 is suitable for use in an alternative embodiment according to the invention, but is not required in order to carry out the invention. Voice activated smart device 28 includes a microphone or an array of microphones for receiving voice commands, where the voice commands initiate action by an operating system. Voice activated smart device 28 is linked to other devices in the network. In this example, voice activated smart device 28 is located in catheterization lab 30, but the device may be located at any point along an associated workflow plan. Clinicians can report patient progress to voice activated smart device 28, including detailed data including images, and/or can request information. Voice activated smart device 28 in turn can upload and transmit data to other devices such as smart watch 10, smart phone 14, or tablet 18, can send alerts and/or push notifications to other devices, such as, for example, smart phone 14. A patient's progress through the workflow can thereby be assisted, tracked, recorded, and analyzed. Examples of voice activated smart devices include, but are not limited to, smart speakers, smart phones, or any device having a voice activated operating system. Well known examples of voice activated operating systems are Ski, Alexa, Echo, and others that are suitable for use according to the invention.

It will be understood that although beacon 16, transmission button 24, and voice activated smart device 28 are illustrated in the examples of FIGS. 3-5 as components of separate systems, any combination of the foregoing elements, including some, all, or none of the aforementioned elements, can be included in a system and/or method according to the invention.

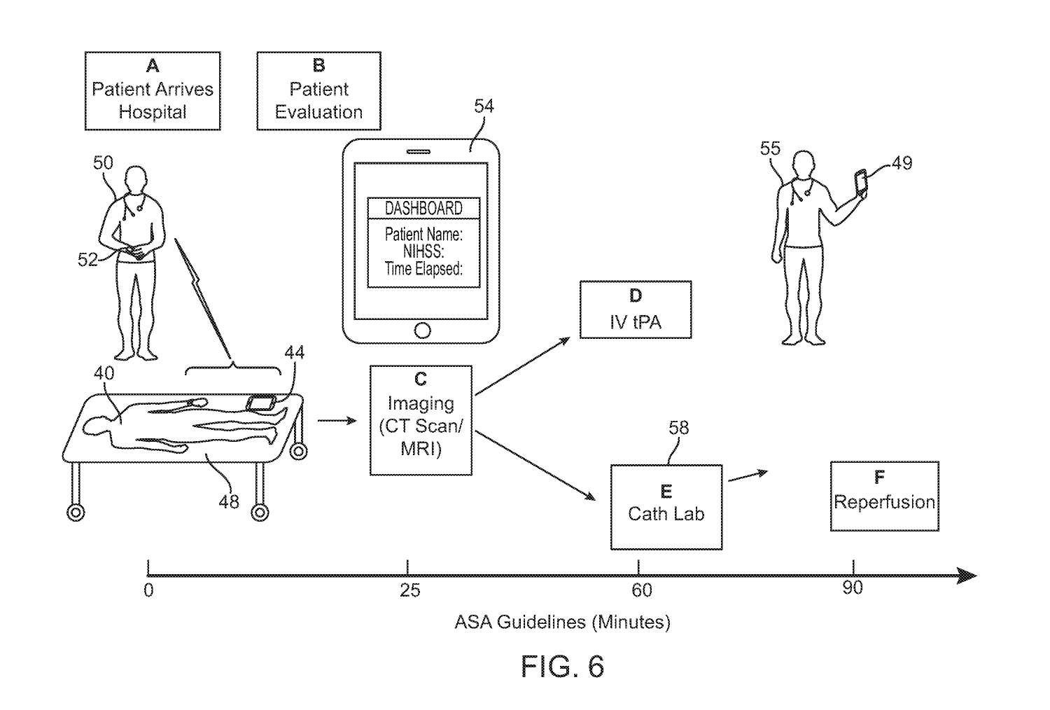

Turning now to FIG. 6, an example of a system and method according to the invention will be illustrated. It will be noted that the embodiment illustrated in FIG. 6 does not require the use of a signal beacon, a transmission button, or a voice activated smart device, though some of the devices in the example may have such capabilities. (For example, smart phone 44 may be equipped with a voice activated operating system such as Ski, but the use of Ski is not required in the embodiment illustrated in FIG. 6.) FIG. 6 highlights conceptual points along a stroke workflow timeline, with the proposed actions illustrated in relation to proposed guidelines, which are displayed along a horizontal axis in the Figure. Beginning at the left hand side of the timeline, representing point A, a stroke patient 40 arrives in the hospital emergency room (ER), at 0 minutes along the timeline, and a smart phone 44 is assigned to the case. Smart phone 44 is configured to send and receive data that correlates to key decisions and actions such as those illustrated in the stroke workflow of FIG. 1.

Further, stroke charge nurse 50 wears smart watch 52. Smart watch 52 is also configured to send and receive communications to other devices, such as smart phone 44, that are relevant to an algorithmic stroke workflow plan, such as that described in FIG. 1. As described above, stroke charge nurse 50 may have received a notification from emergency medical services personnel via smart watch 52. In any event, no later than patient's arrival, stroke charge nurse 50 activates stroke workflow software that is a component of smart watch 52. Smart watch 52 sends a signal to the smart phone 44 to activate a stroke alert app. The ER arrival time is automatically logged, and smart phone 44 is placed on the stretcher 48 in a manner for high visibility of the clock, and for easy access by clinicians. Basic information, such as, for example, a Yes/No selection on a touch screen, may be entered in response to prompts such as “Left side deficit?”, “Right side deficit?”, or “tPA administered?” into smart watch 52, and such inputs will represent progress through a workflow plan. Smart watch 52 and/or smart phone 44 may be otherwise configured so that a clinician can “toggle” through prompts, thereby entering data, and progressing through the stroke workflow. Other basic information, such as patient name, LSW time, NIHSS, neurological deficits, etc. may be entered into either smart watch 52 or smart phone 44. According to the workflow, smart phone 44 sends push notifications and other data, including images, to smart devices utilized by stroke team members (not pictured), and logs the time of associated actions. These push notifications or alerts can be simultaneously transmitted to all other members of the stroke treatment team, thereby enhancing prompt communication to all care providers, and improving timeliness and overall care. In addition, all data can be simultaneously transmitted to and displayed on a dashboard 54 of a smart device.

As the clinicians progress through a workflow plan, patient 40 may be, at point C for example, rolled to the CT scanner 56. The ASA Guidelines highlight the importance of reaching this point in a treatment plan by the time that 25 minutes have elapsed since the patient's arrival, as indicated in FIG. 6. The CT entry time is entered, usually by charge nurse 50, into smart watch 52 and/or smart phone 44, which all the while will be displaying the time lapse, and alerting the app to prompt the next steps. Examples of the data at this point include reporting that the CT is completed, and diagnostic information gleaned from the CT, such as hyperdense sign vs bleed, aspects score, tPA time, whether a large vessel occlusion (LVO) appears, etc. All of the foregoing can be uploaded to an online dashboard and displayed on a device such as dashboard 54. Further, data, images and prompts can be sent to clinicians, such as physician 55, via the clinician's associated smart device, such as smart phone 49. Clinicians can in turn evaluate and act on the data received, such as by initiating a next step in an associated workflow treatment plan, such as the plan illustrated in FIG. 1, or an alternative plan.

If the decision is made to perform a mechanical thrombectomy, the patient is then transported to the cath lab 58. The smart phone 44 remains with patient 40, and upon patient entry into the suite, charge nurse 50 again enters the data into smart watch 52, which then communicates with the phone 44 for logging angiography suite entry time. Cath lab phase data is entered into the phone app via a smart watch 52, which would prompt charge nurse 50 for puncture time, devices used, reperfusion time, TICI score, etc. Upon case completion, all information and images may be immediately available on a dashboard 54 for research/QI or sharing with local EMS and hospital staff. The data can therefore be immediately evaluated, and problem areas within the workflow can be pinpointed for improvement.

Optionally, a system such as the system illustrated in FIG. 6 may optionally include software (not pictured) that is configured to perform machine learning in real time. During performance of a system equipped with such software, the branch points and nodes of a treatment workflow algorithm, such as the workflow illustrated in FIG. 6, may be refined in real time, based on a backend analytics platform. The optional software may include sample data sets (such as age, NIHSS score, etc.) that will trigger particular actions within the workflow, and additionally be modified by accumulating data as the system is utilized. The ongoing analytics may influence the timing of certain actions within the workflow, for example. The performance and success of treatment using the system will, in an ongoing fashion, shape or influence the alerts generated by the system.

It will be understood that the example illustrated in FIG. 6 is greatly simplified for clarity. In an actual clinical setting, numerous clinicians will be members of a stroke team. A stroke team may include, for example, an emergency room physician, a CT scan technician, a neurologist, a neuroradiologist, a neurointerventionalist, a pharmacist, and others. Any and all of the members of the stroke team may be equipped with one or more smart devices linked to an interactive, progressive workflow, which may send prompts to members of the stroke team.

The foregoing examples are not intended to limit the scope of the invention. All modifications, equivalents and alternatives are within the scope of the invention.

What is claimed is:

1. A system for use by a health care provider for diagnosis and treatment of ischemic stroke, the system comprising:
a stroke workflow plan comprising a series of tasks for the diagnosis and treatment of a patient, wherein the series of tasks are organized in an algorithmic fashion;
a first communications device configured with the stroke workflow plan and further configured to receive data and to transmit data corresponding to the tasks in the workflow plan;
a second communications device configured with the stroke workflow plan and further configured to receive data and to transmit data corresponding to the tasks in the workflow plan, whereby progress through the workflow plan proceeds in response to the data received.

2. The system according to claim 1, wherein the first communications device is worn by or is in close proximity to the health care provider during the series of tasks for diagnosis and treatment.

3. The system according to claim 1, wherein the second communications device is worn by or is in close proximity to a patient during the series of tasks for diagnosis and treatment.

4. The system according to claim 1, wherein the series of tasks includes data measurements, and said data measurements are recorded and transmitted by the first communications device.

5. The system according to claim 1, wherein the series of tasks comprises one or more question prompts, and the answers to the questions are recorded and transmitted by the first communications device, and the answers to the questions deploy progress through the workflow in an algorithmic fashion.

6. The system according to claim 1, wherein the system further comprises one or more proximity sensors located geographically at specified checkpoints represented within the workflow plan, wherein the proximity sensors are in wireless communication with the first communications device and are configured to send a location specific signal to the first communications device, whereby the progress of a patient through the workflow plan is tracked and recorded.

7. The system according to claim 1, wherein the second communications device is configured to record and display data received from the first and communications device.

8. The system according to claim 1, wherein the system further comprises one or more manually activated communications device configured to transmit a unique signal to the first communications device or the second communications device or both, wherein the unique signal corresponds to the completion of a task in the stroke workflow plan.

9. The system according to claim 1, wherein the second communications device further comprises a visible timer and an online dashboard.

10. The system according to claim 1, wherein the first communications device or the second communications device or both are configured to receive, to generate, and to transmit one or more prompts, wherein the one or more prompts are associated with one or more tasks that are designated in the workflow plan.

11. The system according to claim 1, wherein the first communications device further comprises one or more voice activated operating systems for receiving and transmitting one or more voice commands, wherein the one or more voice commands correspond to one or more tasks in the workflow plan.

12. The system according to claim 1, wherein the system is configured to automatically upload the data to a software application for review.

13. The system according to claim 1, wherein the first communications device and the second communications device are selected from the group consisting of a smart watch, a smart phone, a laptop computer, a tablet computer, a desktop computer, a smart speaker, and a smart TV.

14. A method of diagnosis and/or treatment of ischemic stroke, the method comprising the steps of:
formulating a work flow plan, the plan comprising a sequence of diagnostic and/or treatment tasks;
providing a first communications device and a second communications device configured with the work flow plan, to receive data and to transmit data in response to the data received, wherein the data received and transmitted is associated with one or more tasks in the work flow plan.

15. The method according claim 14, wherein the first communications device or the second communications device or both are configured to send and receive alert notifications that correspond to one or more tasks in the workflow plan.

16. The method according to claim 14, wherein the first communications device or the second communications device or both comprise a timer, and the the method comprises the additional steps of placing the first communications device on or near a health care provider, and the second communications device on or near a patient, and initiating the timer to track the progress through steps of the work flow plan.

17. The method according to claim 14, wherein the method includes the additional steps of providing a third communications device, wherein the third communications device is figured to receive and display information from the first communications device, the second communications device, or both.

18. The method according to claim 14, wherein the information received by the second communications device from the first communications device comprises the time of administration of a diagnostic test, or the time of administration of a therapeutic treatment, or both.

19. The method according to claim 14, wherein the work flow comprises one or more interventional measures, and the data received by the second communications device from the first communications device comprises elapsed time from a patient's arrival at a health care facility to the time of administration of an interventional measure.

20. The method according to claim 14, wherein the first communications device and the second communications device are selected from the group consisting of a smart watch, a smart phone, a tablet computer, a laptop computer, a smart speaker, and a smart TV.

21. The method according to claim 14, wherein the first communications device is configured to receive question prompts that correspond to one or more tasks in the workflow plan, and the first communications device is further configured to transmit one or more task prompts to the second communications device, wherein the task prompts correspond to one or tasks in the workflow plan.

22. A system for managing a healthcare facility's capability to diagnose and/or treat patients, the system comprising:
a workflow plan comprising a series of tasks for diagnosing and/or treating patients;
a first communications device worn by a patient or placed in close physical proximity to a patient, the first communications device configured to send and to receive data that correspond to one or more tasks of the workflow plan;
a second communications device also configured to send and receive data that correspond to the one or more tasks of the workflow plan;
whereby as the process of diagnosis and/or treatment of the patient progresses through the workflow plan, the data is recorded for analysis.

23. The system according to claim 22, wherein the workflow plan incorporates goal times for completion of one or more tasks in the workflow, and the analysis comprises comparison of actual times of completion of the tasks to the goal times for completion of the tasks.

24. The system according to claim 22, wherein the first communications device is configured to receive question prompts that correspond to one or more tasks of the workflow plan, and the second communications device is configured to receive task prompts, wherein the task prompts correspond to one or more tasks in the workflow plan.

25. The system according to claim 22, wherein the workflow plan comprises goal time for completion of one or more tasks, and the data includes actual elapsed time from the patient's arrival to completion of one or more tasks, and the data is compared to the goal times.

26. The system according to claim 22, wherein one or more of the tasks of the workflow plan comprises diagnostic imaging, data includes arrival at the facility and arrival of the patient at the facility's diagnostic imaging location.

27. The system according to claim 22, wherein the tasks comprise initiation of interventional measures.

28. The system according to claim 22, wherein the system further comprises one or more proximity sensors located within the facility at checkpoints that correspond to one or more tasks in the workflow plan, wherein the proximity sensors are in wireless communication with the first communications device and capable of sending a location specific signal to the first communications device.

29. The system according to claim 22, wherein the second communications device further comprises a visible timer and dashboard that displays data received.

30. The system according to claim 22, wherein the first and second communications devices are selected from the group consisting of: a smart phone, a smart watch, a computer tablet, a laptop computer, a smart TV, a smart speaker, and a desktop computer.

31. The system according to claim 22, wherein the system further comprises one or more manually activated communications devices configured to transmit a unique code to the first communications device, wherein the unique code corresponds to one or more tasks in the workflow plan.

32. The system according to claim 22, wherein the system further comprises a voice activated operating system, wherein the voice activated operating system is configured to send and receive one or more commands that correspond to one or more tasks of the workflow plan.

33. A business method for providing consultation to a stroke treatment facility, the method comprising charging a fee to deploy a system for evaluating the facility's compliance with the American Stroke Association's guidelines for stroke treatment, the system comprising:
a stroke workflow comprising a series of tasks, wherein the tasks correspond to diagnosis or treatment for stroke or both, and the tasks are organized in an algorithmic fashion;
the stroke workflow further comprising goal times for performance of one or more tasks, wherein the goal times correspond to the American Stroke Association guidelines;
a first communications device and a second communications device, both configured to send and to receive data, wherein the data corresponds to the stroke workflow;
whereby the data is compared to the goal times of the American Stroke Association guidelines.

34. The network according to claim 33, wherein the first communications device is configured to transmit one or more prompts to the second communications device.

35. The system according to claim 33, wherein the series of tasks comprises data measurements, and said data measurements are recorded and transmitted by the first communications device.

36. The system according to claim 33, wherein the series of tasks comprises one or more question prompts, and the answers to the questions are recorded and transmitted by the first communications device, and the answers to the questions deploy progress through the workflow algorithm.

37. The system according to claim 33, wherein the system further comprises one or more proximity sensors located geographically at specified checkpoints represented within the workflow plan, the proximity sensors in wireless communication with the first communications device and configured to send a location specific signal to the first communications device, whereby the progress of a patient through the workflow plan is tracked and recorded.

38. The system according to claim 33, wherein the second communications device is configured to record and display data received from the first and second communications devices.

39. The system according to claim 33, wherein the system further comprises one or more manually activated communications device configured to transmit a unique signal to the first communications device or the second communications device or both, wherein the unique signal corresponds to the completion of a task in the stroke workflow plan.

40. The system according to claim 33, wherein the second communications device further comprises a visible timer and an online dashboard.

41. The system according to claim 33, wherein the first communications device or the second communications device or both are configured to receive, to generate, and to transmit one or more prompts, wherein the one or more prompts are associated with one or more tasks that are designated in the workflow plan.

42. The system according to claim 33, wherein the first communications device further comprises one or more voice activated operating systems for receiving and transmitting one or more voice commands, wherein the one or more voice commands correspond to one or more tasks in the workflow plan.

43. The system according to claim 33, wherein the system is configured to automatically upload the data to a software application for review.

44. The system according to claim 33, wherein the first communications device and the second communications device are selected from the group consisting of a smart watch, a smart phone, a laptop computer, a tablet computer, a desktop computer, a smart speaker, and a smart TV.

45. The system according to claim 1, wherein the system further comprises software configured to perform machine learning in real time.
